(19) United States
(12) Patent Application Publication
(10) Pub. No.: US 2021/0222149 A1
(43) Pub. Date: Jul. 22, 2021

(54) METHOD OF ENZYME ENCAPSULATION

(71) Applicant: National Central University, Taoyuan City (TW)

(72) Inventor: Fa-Kuen SHIEH, Taoyuan City (TW)

(21) Appl. No.: 16/837,114

(22) Filed: Apr. 1, 2020

(30) Foreign Application Priority Data
Jan. 20, 2020 (TW) 109101896

(51) Int. Cl. C12N 11/04; B01J 13/04; C12N 11/14; C12N 9/26; C12N 9/08

(52) U.S. Cl. CPC C12N 11/04; B01J 13/04; C12N 11/14; C12N 9/2431; C12Y 111/01006; C12Y 302/01021; C12Y 302/01026; C12Y 302/01023; C12N 9/0065

(57) ABSTRACT

The present disclosure provides a method of enzyme encapsulation, which comprises a step of: mixing an enzyme, a metal-organic framework precursor and a solvent by grinding to encapsulate the enzyme in a metal-organic framework formed by the metal-organic framework precursor, wherein a weight ratio of the enzyme to the metal-organic framework precursor ranges from 1:100 to 1:1, and a weight ratio of the solvent to the metal-organic framework precursor ranges from 1:100,000 to 1:100.

### CROSS REFERENCE TO RELATED APPLICATION

This application claims the benefits of the Taiwan Patent Application Serial Number 109101896, filed on Jan. 20, 2020, the subject matter of which is incorporated herein by reference.

### BACKGROUND OF THE INVENTION

### 1. Field of the Invention

The present disclosure relates to a method of enzyme encapsulation and, more particularly, to a method for encapsulating an enzyme in a metal-organic framework by grinding.

### 2. Description of Related Art

Enzymes are natural catalysts with high specificity, which can accelerate the speed of chemical reactions. It is roughly estimated that the current global enzyme market size is about 5 billion US dollars. At present, the applicable fields of enzymes include food manufacturing and processing (beverage production, sugar production), medicine (artificial red blood cells), health care, beauty, environmental protection (green energy, sewage treatment), crop and animal husbandry, cleaning supplies and other fields.

However, enzymes are often not stable enough in the reaction medium. Studies have found that if the enzyme is encapsulated in a carrier as a biocatalyst, not only its stability and catalytic efficiency can be improved, but also the recovery and reuse of expensive enzymes will become easier, thereby reducing production costs.

In recent years, the metal-organic framework (MOF) has been considered as a suitable solid carrier, because the highly tunable MOF can not only act as an inert carrier, but, also improve the selectivity, stability and activity of enzymes. The so-called “metal-organic framework (MOF)” is a highly crystalline compound complex composed of certain materials and usually has a frame structure. Through different coordination, bonding, and combinations of metals and organic molecules, metal-organic frameworks (MOFs) with different specific properties can be prepared. It is reported that MOF can prevent the entry and exit of chemicals larger than its pore size, thereby giving certain dimensional selectivity; the interaction of enzymes with the MOF can provide enhanced stability to enzymes, for example, the spatial confinement provided by MOF carriers can prevent enzymes from unfolding and losing catalytic activity when it is exposed to denaturing conditions; MOF-encapsulated enzymes have, in certain cases, shown an even higher activity than free enzymes owing to the more-efficient delivery of chemicals induced by the hierarchical porous structure. Further, the MOF synthesis process also allows the introduction of a variety of different enzymes into one MOF structure to run a cascade catalytic reaction.

However, the conventional method of enzyme encapsulation requires the use of elevated temperature and a large amount of organic solvents. Due to the influence of elevated temperature or pH of the solvent, the enzyme conformation is often changed such that the original activity of the enzyme is reduced, the catalytic effect is not as expected, and the synthesis procedure is tedious and time consuming. Therefore, it is urgent to provide a method for encapsulating enzymes to avoid the elevated temperature process, reduce the use of organic solvents, simplify the synthesis steps or shorten the synthesis time.

### SUMMARY OF THE INVENTION

In view of the above, the present disclosure provides a method of enzyme encapsulation, which is simple and fast in synthesis procedure, can be carried out at ambient temperature, or uses few solvents.

In one aspect of the present disclosure, there is provided a method of enzyme encapsulation, comprising a step of: mixing an enzyme, a metal-organic framework precursor and a solvent by grinding to encapsulate the enzyme in a metal-organic framework formed by the metal-organic framework precursor, wherein a weight ratio of the enzyme to the metal-organic framework precursor ranges from 1:100 to 1:1, and a weight ratio of the solvent to the metal-organic framework precursor ranges from 1:100,000 to 1:100.

In the present disclosure, the type of enzyme is not limited and can be selected according to actual needs. In an embodiment of the method according to the present disclosure, the enzyme may be β-glucosidase, invertase, β-galactosidase, catalase or a combination thereof. However, the present disclosure is not limited thereto.

Further, in the present disclosure, the metal-organic framework (MOF) is formed through self-assembling and inter-connection of inorganic metal centers and organic ligands bridged to the inorganic metal centers. Different organic ligands may be used according to different needs, such as imidazole-2-carboxaldehyde, 2-methyl imidazole, imidazole derivatives, or terephthalic acid or derivatives thereof, and the organic ligands bind to inorganic metal ions. Therefore, in the present disclosure, the metal-organic framework precursor used to form the metal-organic framework may include any combination of an organic ligand and a metal ion, which may connect to form the metal-organic framework. In the present disclosure, the metal-organic framework may be a transition metal-based metal-organic framework. For example, the metal-organic framework may be a zinc-based metal-organic framework, a cobalt-based metal-organic framework, a zirconium-based metal-organic framework, a chromium-based metal-organic framework, or other transition metal-based metal-organic frameworks. More specifically, in an embodiment of the method according to the present disclosure, the metal-organic framework precursor may include zirconium (IV) oxo hydroxymethacrylate and 2-aminoterephthalic acid to form a zirconium-based metal-organic framework UiO-66-NH₂. Alternatively, in an embodiment of the method according to the present disclosure, the metal-organic framework precursor may include zinc oxide and 2-methylimidazole to form a zinc-based metal-organic framework ZIF-8. Alternatively, in an embodiment of the method according to the present disclosure, the metal-organic framework precursor may include zinc oxide and 2,5-dihydroxyterephthal c acid to form a zinc-based metal-organic framework Zn-MOF-74. However, it should be understood that the present disclosure is not limited thereto, and those having ordinary skill in the art may select an appropriate metal-organic framework precursor according to the size of the enzyme to be encapsulated.

In an embodiment of the method according to the present disclosure, the grinding may be carried out in a grinding jar at a grinding frequency of 4 to 20 Hz for a grinding time of 0.1 to 10 minutes. Optionally or preferably, the grinding jar is made of zirconium oxide. More preferably, the grinding jar has a volume of 25 ml. However, the present disclosure is not limited thereto. In the present disclosure, the grinding frequency is not limited. Preferably, the grinding frequency ranges from 6 to 12 Hz and, and more preferably, the grinding frequency is around 8 Hz. However, the present disclosure is not limited thereto. In an embodiment of the method according to the present disclosure, the grinding time is preferably 1 to 6 minutes. In another embodiment of the method according to the present disclosure, the grinding time is around 5 minutes. In the present disclosure, a plurality of grinding balls may be contained in the grinding jar for facilitating mixing. Optionally or preferably, the plurality of grinding balls may be made of zirconium oxide. However, the present disclosure is not limited thereto.

Further, in an embodiment of the method according to the present disclosure, optionally or preferably, the step of mixing the enzyme, the metal-organic framework precursor and the solvent by the grinding includes steps of: mixing the solvent and a portion of the metal-organic framework precursor by the grinding for a portion of the grinding time to obtain a mixture; and further adding the enzyme and the rest of the metal-organic framework precursor into the mixture followed by mixing by the grinding for the rest of the grinding time. In one embodiment of the present disclosure, the portion of the metal-organic framework precursor may be 10-90 wt % of the metal-organic framework precursor, or the portion of the grinding time may be 1/10 to 9/10 of the grinding time. More preferably, the portion of the metal-organic framework precursor may be about 50 wt % of the metal-organic framework precursor, or the portion of the grinding time may be about ½ of the grinding time. For example, the solvent and half the metal-organic framework precursor may be mixed by grinding for half the grinding time, followed by addition of the enzyme and the rest of the metal-organic framework precursor and subsequent mixing by grinding for another half of the grinding time. By such procedure, some of the metal-organic framework precursor may be mixed homogeneously first with the aid of the solvent to form the MOF seeds, and the enzyme is added after the solvent has been evaporated to prevent the solvent from destroying the enzyme. In an embodiment of the method according to the present disclosure, the solvent may be methanol, ethanol, dimethyl sulfoxide (DMSO) or a combination thereof. Optionally or preferably, the solvent is ethanol. Moreover, the weight ratio of the solvent to the metal-organic framework precursor is not particularly limited. In an embodiment of the method according to the present disclosure, optionally or preferably, the weight ratio of the solvent to the metal-organic framework precursor ranges from 1:10,000 to 1:1,000; and more preferably, the weight ratio of the solvent to the metal-organic framework precursor ranges from 1:2,000 to 1:1,000. However, the present disclosure is not limited thereto.

In an embodiment of the method according to the present disclosure, the metal-organic framework may be UiO-66-NH₂, ZIF-8, Zn-MOF-74 or any conventional metal-organic framework. Optionally or preferably, the metal-organic framework is UiO-66-NIH, Zit 8, or Zn-MOF-74; and more preferably, the metal-organic framework is UiO-66-NH₂. However, the present disclosure is not limited thereto.

In an embodiment of the method according to the present disclosure, the weight ratio of the enzyme to the metal-organic framework precursor is preferably from 1; 20 to 1:2; more preferably from 1:10 to 1:3; and most preferably about 1:4. However, the present disclosure is not limited thereto.

Furthermore, in an embodiment of the method according to the present disclosure, a purification step may be carried out after the grinding. The purification step may be completed by any conventional purification means, such as centrifugation, washing, drying, etc. so as to facilitate storage for subsequent use. For example, after centrifugation, DI water rinsing may be performed, followed by vacuum drying at room temperature.

The method of the present disclosure may be carried out at a temperature that living organisms survive, such as from 4° C. to 50° C. Generally, it will be relatively simple and convenient to carry out the method of the present disclosure at room temperature. The term “room temperature” used herein may range from 15° C. to 35 DC; and preferably from 25° C. to 28° C. In an embodiment of the method according to the present disclosure, the room temperature is around 25° C.

The method of enzyme encapsulation according to the present disclosure not only has the advantages of simple and rapid synthesis procedure, but also avoids the use of the elevated temperature process and large amounts of solvents, or only a very small amount of solvents is used. Moreover, the solvent may be mixed with a portion of the metal-organic framework precursor before the addition of enzyme so that the solvent may be prevented from contacting the enzyme and destroying the enzyme activity. The pores of the synthesized metal-organic framework have screening capability, so that the destructive molecules larger than the pore size (such as proteolytic enzymes, inhibitors, etc.) can be prohibited from reaching the enzymes, and the enzymes in the framework can fully exert their functions without being interfered. In addition, the enzymes can be immobilized inside the framework to prevent flowing out of the metal-organic framework, to enhance their robustness, and to provide a heterogeneous environment for easy separation. Since the MOFs used as a carrier to encapsulate and immobilize enzymes according to the present disclosure have various pore sizes, they may be combined with various enzymes so as to be more flexible for application in a variety of industrial processes.

Other objects, advantages, and novel features of the disclosure will become more apparent from the following detailed description when taken in conjunction with the accompanying drawings.

### BRIEF DESCRIPTION OF THE DRAWINGS

FIG. 1A shows a PXRD pattern of BGL1@UiO-66-NH₂ and the simulation of UiO-66 according to an embodiment of the present disclosure;

FIG. 1B shows PXRD patterns of BGL2@UiO-66-NH₂ and BGL@ZIF-8 as well as the simulations of UiO-66 and ZIF-8 according to an embodiment of the present disclosure;

FIG. 1C shows PXRD patterns of Inv@UiO-66-NH₂ and β-gal@-66-NH₂ as well as the simulation of UiO-66 according to an embodiment of the present disclosure;

FIG. 1D shows a PXRD pattern of CAT@ZIF-8 and the simulation of ZIF-8 according to an embodiment of the present disclosure;

FIG. 1E shows a PXRD pattern of CAT@Zn-MOF-74 and the simulations of ZnO and MOF-74 according to an embodiment of the present disclosure;

FIG 1F shows a PXRD pattern of ST-BGL@UiO-66-NH₂ and the simulation of UiO-66;

FIG. 2A shows the analysis results by sodium dodecyl sulfate-polyacrylamide gel electrophoresis (SDS-PAGE) according to an embodiment of the present disclosure;

FIG. 2B shows an image of FITC-BGL@UiO-66-NH₂ by confocal fluorescence microscopy according to an embodiment of the present disclosure;

FIG. 2C shows an image of FITC-BGL-on-UiO-66-NH₂ by confocal fluorescence microscopy;

FIG. 3 is a graph showing the kinetics analysis results of BGL1@UiO-66-NH₂, BGL2@UiO-66-NH₂ and ST-BGL@UiO-66-NH₂ according to an embodiment of the present disclosure;

FIG. 4 is a graph showing the comparison of activity between free BGL, BGL@ZIF-8 and BGL2@UiO-66-NH₂ under neutral and acidic conditions according to an embodiment of the present disclosure;

FIG. 5 is a graph showing the kinetics analysis results of Inv@UiO-66-NH₂ according to an embodiment of the present disclosure;

FIG. 6 is a graph showing the kinetics analysis results of β-gal@UiO-66-NH₂ according to an embodiment of the present disclosure;

FIG. 7 is a graph showing the kinetics analysis results of CAT@ZIF-8 according to an embodiment of the present disclosure; and

FIG. 8 is a graph showing the kinetics analysis results of CAT@Zn-MOF-74 according to an embodiment of the present disclosure.

### DETAILED DESCRIPTION OF THE PREFERRED EMBODIMENT

The implementations of the present disclosure will be described with specific embodiments in the following description. A person skilled in the art will understand the advantages and the effects provided by the present disclosure. Different specific embodiments may be applicable according to the present disclosure.

Different embodiments of the present disclosure are provided below. These embodiments are intended to illustrate the technical content of the present disclosure, but not to limit the scope of the present disclosure. A feature of one embodiment can be applied to other embodiments through appropriate modification, replacement, combination, or separation.

Herein, the term “preferably” or “more preferably” is used to describe optional or additional elements or features. In other words, these elements or features are not necessary and may be omitted.

In addition, herein, “about” or “around” a value means a range from the value minus its 10% to the value plus its 10%, and in particular a range from the value minus its 5% to the value plus its 5%.

### Synthesis Example 1

At room temperature, 25 mg (0.0147 mmol) of zirconium (IV) oxo hydroxymethacrylate, 16 mg (0.0882 mmol) of 2-aminoterephthalic acid, and 10 mg of β-glucosidase (BGL) were put into a 25 ml zirconia jar containing 3.5 g of zirconia balls, followed by addition of 41 μl ethanol. The zirconia jar was then set in the Retsch Mixer Mill MM400. After 5 minutes of grinding at 8 Hz, the as-synthesized sample was centrifuged with 30 ml of deionized water at 14,000 g of centrifugal force for three times, washed with 25 ml of deionized water (0° C.) for 1 hour, and finally vacuum dried at room temperature to obtain the final product BGL1@UiO-66-NH₂. The enzyme loading was determined to be about 13.5 wt % by the standard Bradford assay method.

### Synthesis Example 2

At room temperature, 12.5 mg (0.00735 mmol) of zirconium (IV) oxo hydroxymethacrylate and 8 mg (0.0441 mmol) of 2-aminoterephthalic acid were put into a 25 ml zirconia jar containing 3.5 g of zirconia balls, followed by addition of 41 μl ethanol. The zirconia jar was then set in the Retsch Mixer Mill MM400. After 2.5 minutes of grinding at 8 Hz, 12.5 mg (0.00735 mmol) of zirconium (IV) oxo hydroxymethacrylate, 8 mg (0.0441 mmol) of 2-aminoterephthalic acid, and 10 mg of β-glucosidase (BGL) were put into the zirconia jar. After another 2.5 minutes of grinding at 8 Hz, the as-synthesized sample was centrifuged with 30 ml of deionized water at 14,000 g of centrifugal force for three times, washed with 25 ml of deionized water (0° C.) for it hour, and finally vacuum dried at room temperature to obtain the final product BGL2@UiO-66-NH₂. The enzyme loading was determined to be about 15.5 wt % by the standard Bradford assay method.

### Synthesis Example 3

FITC-BGL, a sample of BGL labeled with fluorescent tags, was synthesized as the following. On one hand, 50.0 mg of BGL was dissolved in 2.5 ml of 0.85% physiological saline solution to prepare a BGL solution. On the other hand, a fluorescein-5-isothiocyanate (FITC) solution with a concentration of 10.0 mg/ml was prepared using 0.5 M carbonate-bicarbonate buffer of pH 9.6. Thereafter, 50 μL, of the FITC solution was mixed with the BGL solution and continuously stirred for 30 min to obtain a FITC-BGL solution, and the addition amount of the BGL solution could be adjusted as needed. The resulting FITC-BGL solution was purified using a PD-10 column (50 kDa) and then washed with 0.01 M acetate buffer of pH 5.0 to obtain the FITC-BGL, which was lyophilized and stored at 4° C. for further use.

Afterwards, the synthesis procedures of Synthesis Example 2 were repeated, except that the BGL was replaced with the FITC-BGL to obtain the final product FITC-BGL@UiO-66-NH₂.

### Synthesis Example 4

Zinc oxide (40.7 mg, 0.5 mmol) and 2-methylimidazole (82.6 mg, 1.0 mmol) were prepared and divided into two equal portions. One portion of each was placed in a grinding jar, followed by addition of 60 μL ethanol as the assisting liquid. After 2.5 minutes of grinding at 8 Hz, 11.0 mg of BGL was added, followed by adding the other portion of zinc oxide and 2-methylimidazole. After another 2.5 minutes of grinding at the same grinding frequency, the as-synthesized sample was centrifuged, washed with deionized water for three times, filtered under vacuum, washed with 60 ml of 50% EtOH_{(aq)}, and then vacuum dried at room temperature: to obtain the final product BGL@ZIF-8, which was stored at 4° C. for further use. The enzyme loading was determined to be about 9.5 wt % using the standard Bradford assay method.

### Synthesis Example 5

The synthesis procedures of Synthesis Example 2 were repeated, except that the BGL was replaced with invertase (Inv) to obtain the final product Inv@UiO-66-NH₂. The enzyme loading was determined to be about 14.8 wt % using the standard Bradford assay method.

### Synthesis Example 6

The synthesis procedures of Synthesis Example 2 were repeated, except that the BGL was replaced with β-galactosidase (β-gal) to obtain the final product β-gal @UiO-66-NH₂. The enzyme loading was determined to be about 12.3 wt % using the standard Bradford assay method.

### Synthesis Example 7

Zinc oxide (40.7 mg, 0.5 mmol) and 2-methylimidazole (82.6 mg, 1.0 mmol) were prepared and divided into two equal portions. One portion of each was placed in a grinding jar, followed by addition of 60 μL ethanol as the assisting liquid. After 2.5 minutes of grinding at 8 Hz, 10 mg of catalase (CAT) was added, followed by adding the other portion of zinc oxide and 2-methylimidazole. After another 2.5 minutes of grinding at the same grinding frequency, the as-synthesized sample was centrifuged, washed with deionized water for three times, stirred with 10 ml of proteinase K solution (0.05 mg/ml) for 30 minutes, and vacuum dried at room temperature to obtain the final product CAT@ZIF-8, which was stored at 4° C. for further use. The enzyme loading was determined to be about 2.2 wt % using the standard Bradford assay method.

### Synthesis Example 8

Zinc oxide (36 mg, 0.44 mmol) was mixed with CAT (20 mg) and ml of deionized water in an Eppendorf tube. Thereafter, 2,5-dihydroxyterephthalic acid (44 mg, 0.22 mmol) and 50 μL of Dimethyl sulfoxide (DMSO, 25 vol %) were placed into the grinding jar and ground at a grinding frequency of 15 Hz for 15 minutes. The as-synthesized sample was subsequently centrifuged and quickly washed with 5 ml of deionized water for three times. To remove enzyme residues on the MOF surfaces, the sample was again washed in a 10 ml vial containing 0° C. Tris(hydroxymethyl)aminomethane (Tris) buffer (50 mM, pH 8.0) and proteinase K (0.1 mg/nil), stirred for 30 minutes, and vacuum dried at 25° C. to obtain the final product CAT@Zn-MOF-74, which was stored at 4° C. for further use. The enzyme loading of CAT@Zn-MOF-74 was determined to be about 8.6 wt % by the standard Bradford assay method.

### Comparative Example 1

At room temperature, 12.5 mg (0.00735 mmol) of zirconium (1V) oxo hydroxymethacrylate and 8 mg (0.0441 mmol) of 2-aminoterephthalic acid were put into a 25 ml zirconia jar containing 3.5 g of zirconia balls, followed by addition of 41 μl ethanol. The zirconia jar was then set in the Retsch Mixer Mill MM400. After 2.5 minutes of grinding at 8 Hz, 12.5 mg (0.00735 mmol) of zirconium (IV) oxo hydroxymethacrylates and 8 mg (0.0441 mmol) of 2-aminoterephthalic acid were put into the zirconia jar. After another 2.5 minutes of grinding at 8 Hz, the as-synthesized sample was centrifuged with 30 ml of deionized water at 14,000 g of centrifugal force for three times, washed with 25 ml of deionized water (0° C.) for 1 hour, and finally vacuum dried at room temperature to obtain a MOF product UiO-66-NR). Afterwards, 25 mg of UiO-66-NH₂ was introduced into a 10 ml vial containing 0° C. Tris buffer (50 mM, pH 7.0) and BGL (1.0 mg/ml), stirred for 30 minutes for physical mixing, and vacuum dried at room temperature to obtain the final product BGL-on-UiO-66-NH₂.

### Comparative Example 2

UiO-66-NH₂ (25 mg) was introduced into a 10 ml vial containing 0° C. Tris buffer (50 mM, pH 7.0) and FITC-BGL (1.0 mg/ml), followed by stirring for 10 minutes and vacuum drying at room temperature to obtain the final product FITC-BGL-on-UiO-66-NH₂.

### Comparative Example 3

ZrCl₄ (125 mg) was dissolved in a solution comprising 5 ml of dimethylformamide (DMF) and 1 ml of concentrated HCl. Thereafter, 10 mg of BGL and 134 mg of 2-aminoterephthalic acid in 10 ml of DMF were added, and the mixture was heated in an oven at 80° C. for 10 hours. The as-synthesized sample was centrifuged, washed with 15 ml of DMF for three times, 15 ml of MeOH twice, centrifuged again, and vacuum dried at room temperature to obtain the solvothermal-synthesized MOF composite ST-BGL@UiO-66-NH₂, which was stored at 4° C. for further use. The enzyme loading of ST-BGL@UiO-66-NH₂ was determined to be about 15.1 wt % using the standard Bradford assay method.

### Test Example 1: Detection of MOF Crystal Structures

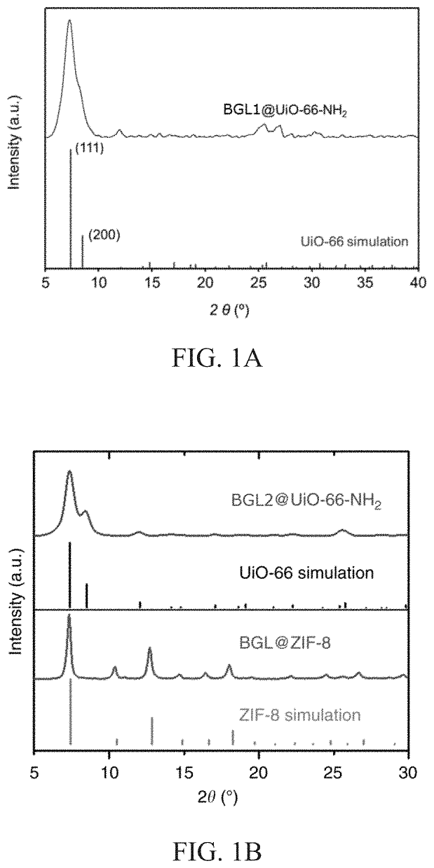

The crystal structures of BGL1@UiO-66-NH₂ of Synthesis Example 1 (FIG. 1A), BGL2@UiO-66-NH₂ of Synthesis Example 2 (FIG. 1B), BGL@ZIF-8 of Synthesis Example 4 (FIG. 1B) Inv@UiO-66-NH₂ of Synthesis Example 5 (FIG. 1C), β-gal@UiO-66-NH₂ of Synthesis Example 6 (FIG. 1C), CAT@ZIF-8 of Synthesis Example 7 (FIG. 1D), CAT@Zn-MOF-74 of Synthesis Example 8 (FIG. 1E), and ST-BGL@UiO-66-NH₂ of Comparative Example 3 (FIG. 1F) were examined by powder x-ray diffraction (PXRD). The appearance of the characteristic peaks of the respective MOF crystals indicated that the introduction of enzymes during the grinding procedure didn't inhibit the formation of MOF crystals.

### Test Example 2: Encapsulation of BGL in UiO-66-NH₂ MOF

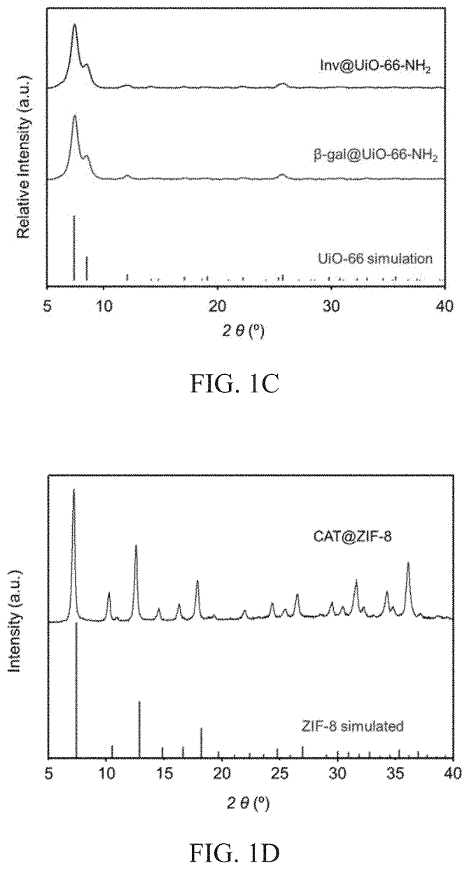

To determine whether the BGL molecules were encapsulated in the UiO-66-NH₂ MOF, in Test Example 2, BGL2@UiO-66-NH₂ of Synthesis Example 2, BGL-on-UiO-66-NH₂ of Comparative Example 1, and ST-BGL@UiO-66-NH₂ of Comparative Example 3 were washed with deionized water, dissolved the covering MOF materials with HCl, and analyzed by sodium dodecyl sulfate-polyacrylamide gel electrophoresis (SDS-PAGE). As shown in FIG. 2A, in which column L1 represented free BGL, column L2 represented BGL-on-UiO-66-NH₂ of Comparative Example 1, and column L3 represented BGL2@UiO-66-NH₂ of Synthesis Example 2, both columns L1 and L3 clearly showed a bond corresponding to the molecular weight of monomeric BGL, 65 kDa, indicating that BGL was indeed encapsulated in UiO-66-NH₂ MOF during the grinding process for BGL2@UiO-66-NH₂ and couldn't be removed by washing, but no band was observed in column L2, indicating that BGL was only adsorbed on the external surface of UiO-66-NH₂ MOF in BGL-on-UiO-66-NH₂ and the adsorption force was not strong such that BGL was removed after washing.

Another test was carried out to demonstrate the encapsulation of BGL molecules in UiO-66-NH₂ MOF. To reveal the distribution of enzymes, FITC-BGL@UiO-66-NH₂ of Synthesis Example 3 and FITC-BGL-on-UiO-66—NR₂ of Comparative Example 2 were examined by confocal microscopy. The fluorescence image of FITC-BGL@UiO-66-NH₂ showed that the BGL molecules were distributed evenly throughout the UiO-66-NH₂ MOF (FIG. 2B), indicating that the BGL molecules were indeed encapsulated in the UiO-66-NH₂ MOF in FITC-BGL@UiO-66-NH₂. However, the fluorescence image of FITC-BGL-on-UiO-66-NH₂ showed that the BGL molecules were only distributed on the surface of the UiO-66-NH₂ MOF (FIG. 2C), confirming that the BGL molecules were only adsorbed on the surface of the UiO-66-NH₂ MOF to FITC-BGL-on-UiO-66-NH₂.

### Test Example 3: Activity of BGL@UiO-66-NF₂

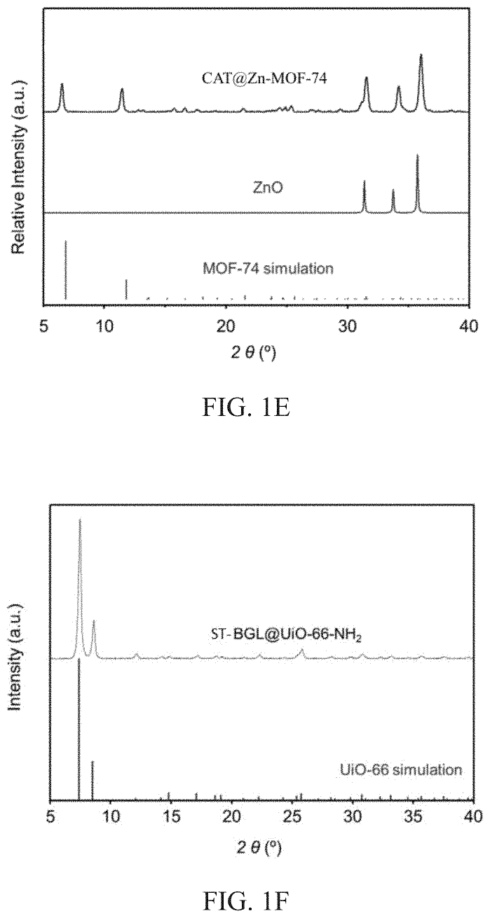

The hydrolysis of one of cellobiose's analogs, 4-nitrophenyl β-D-glucopyranoside (pNPG), to 4-nitrophenol (pNP) was carried out to determine the reaction activity of encapsulated BGL. The samples of Synthesis Example 1 Synthesis Example 2 and Comparative Example 3 were dispersed into 0.5 ml of buffer (pH 6.0, 20 mM), respectively. After incubation at 37° C. for 30 minutes, the reaction was run by adding 0.5 ml of 4 mM pNPG, keeping for the desired period of time, and then pipetting 50 μl of the solution into 950 ul of NaOH-glycine buffer (0.4 M, pH 10.8) to terminate the reaction. The absorbance at 405 nm of pNP is monitored as a measure of reaction activity. As shown in FIG. 3, the observed rate constant was determined for the samples of Synthesis Example 1 (k_{obs}=2.8×10⁻⁴ s⁻¹) and Synthesis Example 2 (k_{obs}=5.0×10⁻⁴ s⁻¹), indicating that the method of enzyme encapsulation of the present disclosure allows enzymatic functionality to be preserved. As expected, the sample of Comparative Example 3 showed no biological activity.

### Test Example 4: Protection Functionality of MOF

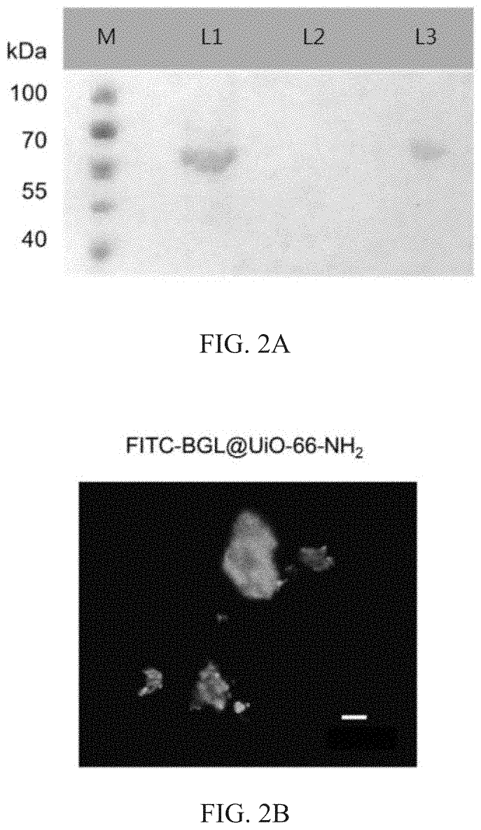

Protease can hydrolyze peptide bonds and deactivate BGL and, therefore, the hydrolysis of pNPG was carried out in the presence of protease to test the protection functionality of MOF on BGL. The protease used herein is a mixture of three proteolytic enzymes, with size ranging from 16 kDa to 27 kDa, to ensure hydrolysis efficiency. Three samples, i.e. free BGL, BGL2@UiO-66-NH₂ of Synthesis Example 2, and BGL@ZIF-8 of Synthesis Example 4, were dispersed into 0.5 ml of buffer (pH 6.0, 20 mM). After incubation at 37° C. for 30 min, the activity was assessed by adding 0.5 ml of 4 mM pNPG, letting the reaction run for the desired amount of time, and then terminating the reaction by pipetting 50 μL of the solution into 950 μl, of a NaOH-glycine buffer (0.4 M, pH 10.8). As shown in FIG. 4, free BGL exhibited fully catalytic activity under acidic conditions (pH 6.0) but retained only 16% of its activity after protease exposure. However, ZIF-8 is unstable under acidic conditions (pH 6.0). As ZIF-8 breaks down under acidic conditions, BGL is exposed and hydrolyzed by protease, so that BGL@ZIF-8 shows lower biological activity (˜40%) after protease exposure. Although ZIF-8 is stable in a neutral environment, pNPG cannot pass through the pores (˜3.5 Å) of ZIF-8 to react with BGL inside ZIF-8 due to its bigger size (5.4 Å×6.0 Å), In contrast, the activity of BGL2UiO-66-NH₂ showed no significant decay after a 2 h incubation with protease (pH 6.0) because UiO-66-NH₂ is stable under acidic conditions and protects BGL from protease, which is much larger than the pore size (6.0 Å) of UiO-66-NH₂. Also, pNPG (5.4 Å×6.0 Å) is allowed to pass through the pores (6.0 Å) of UiO-66-NH₂, to react with BGL therein.

### Test Example 5: Activity of Inv@UiO-66-NH₂

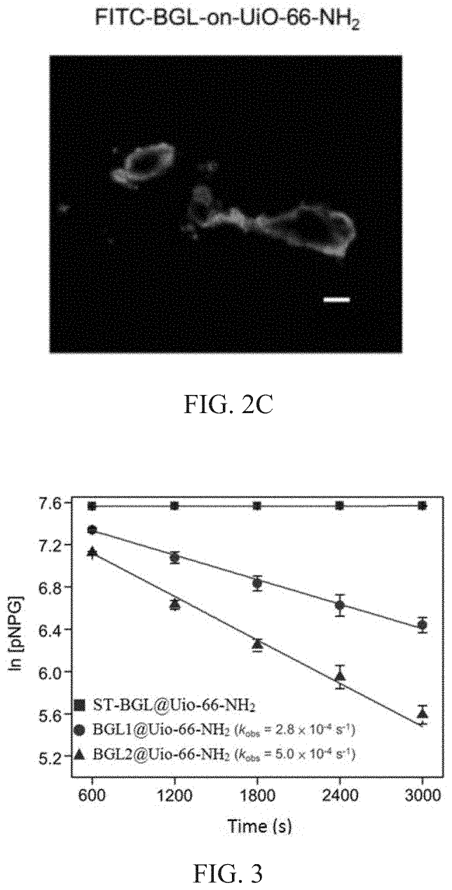

For Inv@UiO-66-NH₂, 2.0 mg of Inv@UiO-66-NH₂ from Synthesis Example 5 (˜14.8+1.2 wt % of Inv) were dispersed into 0.5 ml of a citric buffer (pH 4.4, 20 mM) and incubated at 37° C. for 30 min. The biological activity of Inv was assayed through the addition of 0.5 ml of 4 mM sucrose (citric buffer solution) as substrate. After a period of reaction time, the reaction was terminated by pipetting 50 UL of the reaction solution into 950 μL of PAHBAH reagent (5 mg/ml PAHBAH in 0.5 M NaOH), which was then heated at 95° C. for 6 min, cooled at 4° C. for 1 min, and reheated at room temperature for 1 min, and the absorbance at 410 nm was read. The observed rate constant k_{obs} was determined to be 2.0×10⁻³ s⁻¹, as illustrated in FIG. 5.

### Test Example 6: Activity of β-gal@UiO-66-NH₂

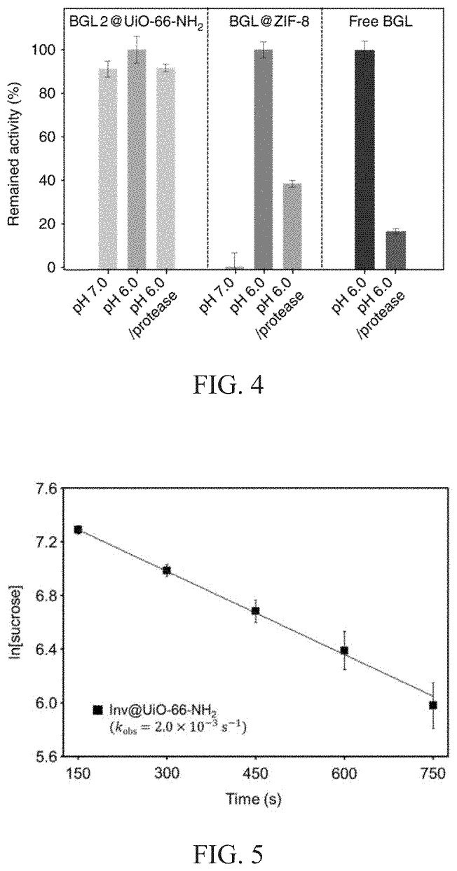

For determining the activity of β-gal@UiO-66-NH₂, ˜0.6 mg of β-gal@UiO-66-NH₂ from Synthesis Example 6 (˜12.5 wt % of β-gal) was dispersed in 0.5 ml citric buffer (pH 5.0, 201′11\4) and incubated at 37° C. for 30 min, followed by adding 0.5 ml of 5 mM 2-nitrophenyl β-D-galactopyranoside (oNPG) (citric buffer solution) as a substrate, and the reaction was subsequently terminated by pipetting 50 μL of solution into 950 μL of Na₂CO₃ (1.0 M). The catalytic activity of β-gal was determined from the concentration of 2-nitrophenol (oNP), which was calculated by measuring the absorbance at 417 nm using a Jasco V-730 ultraviolet-visible spectrophotometer. The observed rate constant k_{obs} was determined to be 1.1×10⁻⁴s⁻¹, as illustrated in FIG. 6.

### Test Example 7: Activity of CAT@ZIF-8

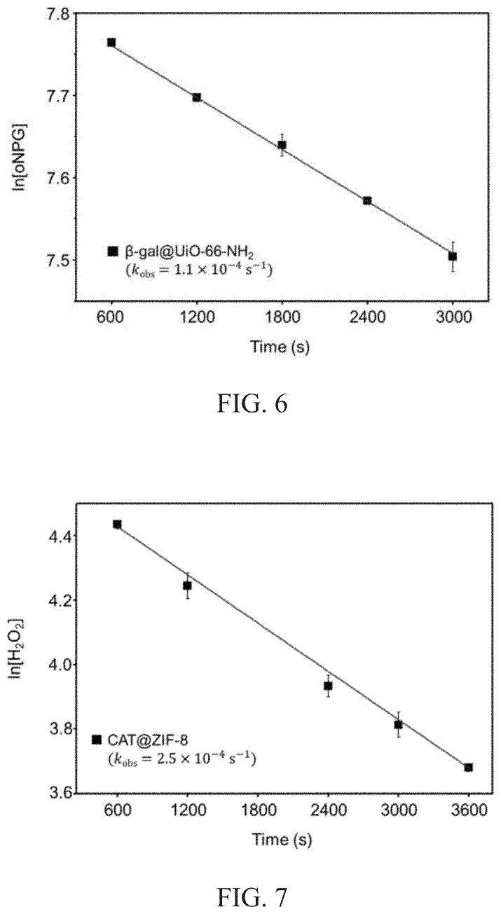

It is known that catalase (CAT) can decompose hydrogen peroxide to water and oxygen. Therefore, degradation kinetics of hydrogen peroxide were studied to evaluate the biological activity of the catalases embedded into the metal-organic framework (MOF) prepared via the method of present disclosure. By using FOX assay, iron divalent ions (Fe²⁺) of the FOX reagent (including ferrous ammonium sulfate, sorbitol, sulfuric acid and xylenol orange) will react with the remaining hydrogen peroxide and become iron trivalent ions (Fe³⁺). Then, under slightly acidic condition, the iron trivalent ions (Fe³⁺) and xylenol orange will form complexes, which exhibit good linear absorption intensity at UV-Vis 560 nm with respect to its concentration. Thereby, the concentration of the remaining hydrogen peroxide can be obtained indirectly. In this Test Example, Catalase (CAT) was chosen to be encapsulated into ZIF-8 as it catalyzes hydrogen peroxide dissociation and hydrogen peroxide Is smaller than the ZIF-8 pore size, allowing hydrogen peroxide to reach CAT. To demonstrate that ZIF-8 could protect CAT, 13.6 mg of CAT@ZIF-8 (˜2.2 wt % CAT in CAT@ZIF-8) from Synthesis Example 7 was incubated in 400 μL of 50 mM Tris buffer (pH 8.0) for 30 min and subsequently added to 100 μL of 50 mM Tris buffer (pH 8.0) with 0.05 mg proteinase K to incubate for 1 h. It should be noted that proteinase K has a molecular size of 68.3×68.3×108.5 Å (28.5 kDa), which is greater than the pore size of ZIF-8. The activity was determined by adding 500 μL of 200 μM H₂O₂ in pH 8 Tris buffer solution. The biological activity assays showed an observed rate constant (k_{obs}) of 2.5×10⁻⁴ s⁻¹, as shown in FIG. 7, demonstrating that CAT encapsulated in ZIF-8 retain their biological activity, and ZIF-8 possess size selectivity to protect CAT from proteinase K.

### Test Example 8: Activity of CAT@Zn-MOF-74

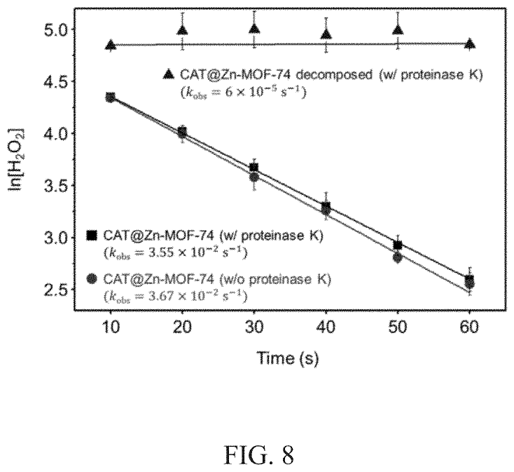

To demonstrate the generality of the method, CAT was encapsulated in Zn-MOF-74 via the grinding process. Zn-MOF-74 a member of the M-MOF-74 (CPO-27) family, was formed with stoichiometric ZnO and 2,5-dihydroxyterephthalic acid (H4dhta). Prior to the biological activity assay, CAT@Zn-MOF-74 of Synthesis Example 8 was incubated in pH 8.0 Tris buffer with proteinase K to remove residual CAT on the Zn-MOF-74 surface. 3.5 mg of the washed CAT@MOF-74 (˜8.6 wt % CAT) was incubated in 400 μL of 50 mM Tris buffer (pH 8.0) for 30 min and then added into 100 μL of 50 ml Tris buffer (pH 8.0) containing proteinase K (1.0 mg/ml) for 30 min. The solution was assayed by addition of 500 μL, of 200 mM H₂O₂ in pH 8 Tris buffer solution. As illustrated in FIG. 8, CAT@Zn-MOF-74 without proteinase K incubation showed an observed rate constant of 3.67×10⁻²s⁻¹. However, CAT@Zn-MOF-74 with proteinase K incubation exhibited a similar high observed rate constant of 3.55×10⁻² s⁻¹, indicating the protection functionality of Zn-MOF-74, Due to the size selectivity of ZIT-IMF-74, CAT was protected from proteinase K and retained its biological activity. As to the control group, in which CAT@Zn-MOF-74 was decomposed using 1.875 M of NaOH (pH˜8.0) and subsequently incubated in Tris buffer (pH 8.0) containing proteinase K (1.0 mg/ml) for 30 min, the observed rate constant was only 6×10⁻⁵s⁻¹ due to the decomposition of Zn-IMF-74 and the inhibition of CAT by proteinase K.

In summary, the method of the present disclosure is simple and fast in synthesis procedure, can be carried out at ambient temperature, can reduce the use of solvents or only use few solvents, and can effectively maintain the activity of enzymes and protect enzymes from the destruction of macromolecular compounds (such as proteases). The method of the present disclosure can be generally applied to encapsulate enzymes of various sizes into MOB having different pore sizes, so as to be widely used in various industries.

Although the present disclosure has been explained in relation to its preferred embodiment, it is to be understood that many other possible modifications and variations can be made without departing from the spirit and scope of the disclosure as hereinafter claimed.

What is claimed is:

1. A method of enzyme encapsulation, comprising a step of: mixing an enzyme, a metal-organic framework precursor and a solvent by grinding to encapsulate the enzyme in a metal-organic framework formed by the metal-organic framework precursor, wherein a weight ratio of the enzyme to the metal-organic framework precursor ranges from 1:100 to 1:1, and a weight ratio of the solvent to the metal-organic framework precursor ranges from 1:100,000 to 1:100.

2. The method of claim 1, wherein the enzyme includes β-glucosidase, invertase, β-galactosidase, catalase or a combination thereof.

3. The method of claim 1, wherein the metal-organic framework precursor includes zirconium (IV) oxo hydroxymethacrylate and 2-Aminoterephthalic acid.

4. The method of claim 1, wherein the metal-organic framework precursor includes zinc oxide and 2-methylimidazole.

5. The method of claim 1, wherein the metal-organic framework precursor includes zinc oxide and 2,5-dihydroxyterephthalic acid.

6. The method of claim 1, wherein the solvent includes methanol, ethanol, dimethyl sulfoxide (DMSO) or a combination thereof.

7. The method of claim 1, wherein the weight ratio of the solvent to the metal-organic framework precursor ranges from 1:10,000 to 1:1,000.

8. The method of claim 1, wherein the grinding is carried out in a grinding jar at a grinding frequency of 4 to 20 Hz.

9. The method of claim 8, wherein the grinding jar is made of zirconium oxide.

10. The method of claim 8, wherein a plurality of grinding balls are contained in the grinding jar.

11. The method of claim 10, wherein the plurality of grinding balls are made of zirconium oxide.

12. The method of claim 8, wherein the grinding frequency ranges from 6 to 12 Hz.

13. The method of claim 1, wherein the grinding is carried out for a grinding time of 0.1 to 10 minutes.

14. The method of claim 13, wherein the grinding is carried out for the grinding time of 1 to 6 minutes.

15. The method of claim 13, wherein the step of mixing the enzyme, the metal-organic framework precursor and the solvent by the grinding includes steps of: mixing the solvent and a portion of the metal-organic framework precursor by the grinding for a portion of the grinding time to obtain a mixture; and further adding the enzyme and the rest of the metal-organic framework precursor into the mixture followed by mixing by the grinding for the rest of the grinding time.

16. The method of claim 15, wherein the portion of the metal-organic framework precursor is 10-90 wt % of the metal-organic framework precursor.

17. The method of claim 15, wherein the portion of the grinding time is 1/10 to 9/10 of the grinding time.

18. The method of claim 1, wherein the metal-organic framework is UiO-66-NH₂, ZIF-8, or Zn-MOF-74.

19. The method of claim 1, wherein the weight ratio of the enzyme to the metal-organic framework precursor ranges from 1:20 to 1:2.

20. The method of claim 19, wherein the weight ratio of the enzyme to the metal-organic framework precursor ranges from 1:10 to 1:3.
